(19) United States
(12) Patent Application Publication
(10) Pub. No.: US 2005/0080391 A1
(43) Pub. Date: Apr. 14, 2005

(54) Absorbent article

(75) Inventors: Wataru Yoshimasa, Kagawa (JP); Kazuya Nishitani, Kagawa (JP); Noritatsu Tamagawa, Kagawa (JP)

Correspondence Address: DARBY & DARBY P.C., P. O. BOX 5257, NEW YORK, NY, 10150-5257, US

(73) Assignee: Uni-Charm Corporation, Shikokuchuo-Shi (JP)

(21) Appl. No.: 10/959,753

(22) Filed: Oct. 6, 2004

Related U.S. Application Data
(63) Continuation of application No. PCT/JP03/10493, filed on Aug. 20, 2003.

(30) Foreign Application Priority Data
Aug. 30, 2002 (JP) 2002-253820

(51) Int. Cl. A61F 13/15; A61F 13/20

(52) U.S. Cl. 604/385.01

(57) ABSTRACT

An absorbent article which can effectively widen absorbing area of excretion liquid at a center portion of the absorbent article, is provided. Divided absorbent elements are formed dividingly on both of a center line. The divided absorbent elements are fixed on a back sheet at positions of compressed portions, and inner side end portions of the divided absorbent elements are taken as free ends. Upon use of a sanitary napkin, by bending the sanitary napkin in response to exertion of compressing force F from femoral parts, the divided absorbent elements are raised so that menstrual blood can be absorbed not only through inner surfaces but also through outer surfaces. Therefore, absorbing area can be greater than that of the conventional sanitary napkin, and side leakage can be successfully prevented.

### BACKGROUND OF THE INVENTION

1. Field of the Invention

The present invention relates generally to an absorbent article to be used as a sanitary napkin, a panty liner for absorbing lady's vaginal discharge, an incontinence pad, an absorbent assist pad for diaper, a disposable diaper or the like. More particularly, the invention relates to an absorbent article which can effectively widen absorbing area of excretion liquid as compared with the prior art and easily prevent side leakage of the liquid.

2. Description of the Related Art

Absorbent article used for absorbing lady's menstrual blood is desired to have large liquid absorbing capacity with high liquid absorbing speed, not to easily cause side leakage of liquid, and to be firmly fitted to a discharging part of body fluid.

For example, Japanese Unexamined Patent Publication No. Heisei 9-313529 discloses an absorbent article formed with a predetermined width of ridge at a center portion of a surface on a liquid receiving side. The ridge is formed by folding the center portion of an absorbent sheet appearing on the surface side of the absorbent article, bulging toward the surface side. By providing substantially wider surface area by folding the center portion of the absorbent sheet for bulging, the absorbent article may increase absorbing amount of body fluid at the center portion thereof. Also, by firmly fitting the ridge onto labial portion, the excretion liquid is directly applied to the portion of the ridge for preventing side leakage of excretion liquid.

It should be noted that throughout the disclosure, claims and abstract, “inner surface” of the absorbent article means a surface mating a skin of user, “inner surface side” indicates a side of the inner surface of an absorbent body of the absorbent article, “outer surface” means a surface remote from the skin of user and exposed outwardly or mated with a crotch portion of a sanitary panty, “outer surface side” indicates a side of the outer surface of the absorbent body, “longitudinal direction” of the absorbent article is a direction corresponding to back and force direction of a body of user as fitted on the user, and “lateral direction” is a direction perpendicular to the longitudinal direction and corresponding to right and left direction of the body of user.

However, in the absorbent article, since a rest portion of the absorbent sheet is continuously located with the ridge, when large amount of excretion liquid is excreted and applied to the absorbent article, the excretion liquid may flow toward a peripheral edge portion of the absorbent article as guided by the absorbent sheet rather than permeating through the absorbent sheet. Accordingly, it is impossible to sufficiently achieve an effect of prevention of side leakage of the excretion liquid.

On the other hand, Japanese Unexamined Patent Publication No. Heisei 9-313530 discloses an absorbent article having a structure, in which an absorbent substrate provided below a water permeable surface sheet is folded toward a back sheet in recessed manner at a center portion so that both of left and right side portions are constituted of a single sheet and the center portion is constituted of three stacked sheets. In the above-identified publication, as an effect of the absorbent article, the excretion liquid is guided into a recessed portion at the center portion of the absorbent substrate to be absorbed into the absorbent substrate. On the other hand, since greater number of sheets are stacked at the center portion, absorbing capacity can be increased as compared with the conventional absorbent article to reduce necessity of prevention of side leakage.

However, since the foregoing absorbent article is covered the surface of the folded absorbent substrate with the flat water permeable surface sheet, the excretion liquid applied to the surface sheet may laterally flow toward side edges along the surface sheet before being absorbed by the absorbent substrate to easily cause side leakage. Furthermore, since the absorbent substrate has a structure to be simply wrapped with a leakage preventing sheet on back side and the surface sheet in the folded condition, when the absorbent article is used with fitting on inside of an underwear and a pressure is exerted therein from femoral regions on both sides, the center portion may not always be deformed for firmly fitting on an excretion portion of the wearer's body. As a result, difficulty can be encountered in assuring firm fitting onto the excretion portion, and the excretion liquid applied to the surface sheet can easily cause side leakage.

Next, Japanese Unexamined Utility Model Publication No. Heisei 4-128728 discloses an absorbent article having left and right absorbent body portions divided into two and respectively extending in longitudinal direction. As shown in FIG. 19, an absorbent body 100 provided on a back sheet (leakage preventing sheet) 106 is divided into two absorbent body portions 100a and 10b which are separated away from each other. Two absorbent body portions 100a and 10b are covered with a surface sheet 101 over the entire area. At both end portions in longitudinal direction having a section shown in FIG. 19, the surface sheet covering laterally inner side end portions of the absorbent body portions 100a and 100b are bonded at bonding portions 102. In a portion corresponding to wing portions in longitudinal direction, the surface sheet covering laterally inner side end portions of the absorbent body portions 100a and 100b are abutted in non-bonded condition. In a not fitted condition, the portion corresponding to the wing portions have similar section as shown in FIG. 19. In a fitted condition, when the wing portions are bent outwardly, the surface sheet covering laterally inner side end portions of the absorbent body portions 100a and 100b are raised to be placed in mutually spaced apart relationship to form an open portion. Also, at abutting portion in the vicinity of a leakage preventing sheet, the surface sheet covering laterally inner side end portions of the absorbent body portions 100a and 100b are abutted with each other in non-bonded condition. It should be noted that the surface sheet located on the side of the leakage preventing sheet of the abutting portion are directly bonded on the leakage preventing sheet to form a menstrual blood receptacle portion.

In the foregoing prior art, in the condition where the absorbent article is fitted on the user's body, the open portion defined by raised absorbent body portions 100a and 100b and the surface sheet 101 guides menstrual blood into the menstrual blood receptacle portion to facilitate absorption thereof in the absorbent body portions 100a and 100b, and whereby prevents the menstrual blood from spreading along the surface sheet 101.

However, in the structure of the absorbent article shown in FIG. 19, the surface sheet covering laterally inner side end portions of the absorbent body portions 100a and 100b cannot be raised as proposed but are collapsed as shown in FIG. 20 to cause difficulty in guiding the menstrual blood into the menstrual blood receptacle portion. Also, difficulty may be encountered in firmly fitting the portion of the surface sheet covering laterally inner side end portions of the absorbent body portions 100a and 100b defining the open portion to the excretion portion of the user's body.

FIG. 20 shows a condition where the absorbent article shown in FIG. 19 is fitted on crotch part as bonded on a sanitary panty 103. In the fitted condition, lateral compressing forces F are exerted on both side edge portions 104 and 105 of the absorbent article from femoral parts to cause compressing deformation of the absorbent article so that distance between both side edge portions 104 and 105 can be reduced. In the shown absorbent article, since both of the absorbent body portions 100a and 100b are sandwiched between the back sheet 106 and the surface sheet 101, when the lateral compressing forces F are applied on the absorbent body portions 100a and 10b, such lateral compressing forces F mainly act for causing compressing deformation of the absorbent body portions 100a and 100b and do not act for raising the absorbent body portions 100a and 100b toward the user's body.

Accordingly, as shown in FIG. 20, when the absorbent body portions 100a and 100b are collapsed by the lateral compressing forces F to block the open portion, it becomes difficult to achieve initially expected function to guide the menstrual blood into the menstrual blood receptacle portion to absorb in respective absorbent body portions 100a and 100b. Also, difficulty may be encountered in firmly fitting the portion of the surface sheet covering laterally inner side end portions of the absorbent body portions 100a and 100b defining the open portion to the excretion portion of the user's body. Therefore, it is difficult to prevent spreading of menstrual blood.

### SUMMARY OF THE INVENTION

The present invention has been worked out in view of the drawbacks in the prior art. It is therefore an object of the present invention to provide an absorbent article to effectively widen absorbing area of excretion liquid particularly in a center portion, to facilitate firmly fitting the center portion onto an excreting portion of a user's body, and to hardly cause fear for side leakage of excretion liquid.

An absorbent article provided with an absorbent element absorbing and holding a liquid, on a flat base and formed into a longitudinally elongated shape, comprising:
a cushion layer forming the base;
divided absorbent elements forming the absorbent element and dividedly formed on both sides of a center line extending in a longitudinal direction of the absorbent article;
inner side end portions of respective divided absorbent elements located in opposition across the center line; and
compressed portions located at positions away from the center line, formed by compressing the divided absorbent elements and the cushion layer together and having portions extended continuously or aligned in the longitudinal direction;
each divided absorbent element being deformable to move the inner side end portions away from the cushion layer about the compressed portions.

The absorbent article has the divided absorbent elements provided on both sides and fixed by compression together with the cushion layer at positions away from the center line, and the divided absorbent elements are independent with each other so that the inner side end portions directed to the center line become free ends.

Therefore, when the absorbent article is fitted on wearer's body, the divided absorbent elements are biased to raise toward the wearer's body about the compressed portion. Furthermore, since the cushion layer is deformed to curve upon fitting, the divided absorbent elements are effectively raised toward the wearer's body. Accordingly, the divided absorbent elements can be easily and firmly fitted on the excretion portion of the wearer's body. Also, a gap is defined between a pair of the inner side end portions of the divided absorbent elements to introduce the excretion liquid to ensure absorption by the divided absorbent elements, and to successfully prevent side leakage.

In the preferred embodiment of the present invention, each divided absorbent element may have a divided absorbent layer and a liquid permeable surface sheet covering the divided absorbent layer.

Since the individual divided absorbent element is independently covered by the surface sheet, the excretion liquid can be transferred to an outer surface of the divided absorbent element directed to the cushion layer to ensure absorption of the excretion liquid by each divided absorbent elements.

It should be noted that the divided absorbent elements are not limited to those in which the divided absorbent layer is independently covered by the surface sheet, but can be covered with common surface sheet.

The present invention can be constructed with the cushion layer formed with an absorbent layer absorbing and holding the liquid.

When the cushion layer functions as the absorbent layer, menstrual blood penetrating between the inner side end portions of respective divided absorbent elements is absorbed by the cushion layer and is prevented from exuding from the back or outer surface of the absorbent article.

In the preferred construction of the present invention, the compressed portion may be formed surrounding a predetermined area of the absorbent article.

When the compressed portion is the pattern surrounding the predetermined area, the excretion liquid applied to the portion surrounded by the compressed portion is difficult to disperse to the periphery beyond the portion surrounded by the compressed portion.

Also, in the preferred construction of the present invention, longitudinally extending elastic members are provided in respective divided absorbent elements.

In case that the longitudinally extending elastic members are provided, when the absorbent article is bowed by the compressing force from both sides, the divided absorbent elements are easily raised about the compressed portion so as to direct the inner side end portions toward the wearer's body.

Furthermore, in the preferred construction of the present invention, respective divided absorbent elements are bent to place the inner side end portions toward inner surface side at positions between the inner side end portions and the compressed portion.

In this construction, at the center portion of the absorbent article, the absorbent layer can be made substantially thick to increase absorbing capacity of the excretion liquid.

Furthermore, in the preferred construction of the present invention, a thinned portion is provided in the cushion layer along the center line.

With this construction, when compressing force is applied from femoral parts on both side portions, the absorbent article is easily deformed to direct the center portion toward the wearer's body, and the divided absorbent elements are firmly fitted on the excretion portion of the wearer's body.

Furthermore, in the preferred construction of the present invention, each divided absorbent layer is formed with a plurality of laterally extending absorbent layers.

When the divided absorbent layer is divided into a plurality of absorbent layers aligned in longitudinal direction, even when the absorbent article fitted on the crotch part is deformed to curve so that the front edge portion and the rear edge portion approach with each other, the divided absorbent elements are hardly twisted in the condition deformed toward the excretion portion of the wearer's body.

On the other hand, the absorbent article according to the present invention may be a sanitary napkin.

Since the absorbent article according to the present invention can absorb the excretion liquid by the entire surface of respective divided absorbent elements by introducing the excretion liquid between the inner side end portions of the left and side divided absorbent elements, side leakage can be prevented in lateral direction. Therefore, the absorbent article according to the present invention is particularly preferred as the sanitary napkin. In case of the sanitary napkin, since the menstrual blood can be absorbed effectively by the divided absorbent elements, it can be a structure having high side leakage preventing effect even when the divided absorbent elements are thin and the cushion layer is thin. Accordingly, thin and small size sanitary napkin can be easily constructed.

Furthermore, because of the structure easily absorbing menstrual blood by the divided absorbent element, amount of absorbing material, such as superabsorbent polymer, pulp or the like to be contained in the divided absorbent element, can be made smaller. Furthermore, since side leakage preventing effect is high, it becomes possible to eliminate the leakage preventing walls having gather, and even when the leakage preventing walls are provided, the leakage preventing walls can be made smaller or thinner. Therefore, since amount of the pulp or superabsorbent polymer can be reduced or the leakage preventing walls can be formed in smaller dimension or of expensive material while liquid blocking performance being lower, production cost can be reduced.

### BRIEF DESCRIPTION OF THE DRAWINGS

The present invention will be understood more fully from the detailed description given hereinafter and from the accompanying drawings of the preferred embodiment of the present invention, which, however, should not be taken to be limitative to the invention, but are for explanation and understanding only.

In the drawings:

FIG. 1 is a perspective view showing a sanitary napkin as the first embodiment of an absorbent article according to the present invention;

FIG. 2 is a cross-section taken along line II-II of FIG. 1;

FIG. 3 is an enlarged section of right half of FIG. 2;

FIG. 4 is a section showing a condition where the sanitary napkin shown in FIG. 1 is deformed upon fitting on a user's body;

FIG. 5 is a section showing a sanitary napkin as the second embodiment of an absorbent article according to the present invention;

FIG. 6 is a partial section of the third embodiment of a sanitary napkin according to the present invention;

FIG. 7 is a plane view of the fourth embodiment of a sanitary napkin according to the present invention;

FIG. 8 is a partial section taken along line VIII-VIII of FIG. 7;

FIG. 9 is a plane view of the fifth embodiment of a sanitary napkin according to the present invention;

FIG. 10 is a partial section taken along line IX-IX of FIG. 9;

FIG. 11 is a partial section of the sixth embodiment of a sanitary napkin according to the present invention;

FIG. 12 is a partial section of the seventh embodiment of a sanitary napkin according to the present invention;

FIG. 13 is a section showing a condition where the sanitary napkin shown in FIG. 12 is deformed upon fitting on a user's body;

FIG. 14 is a perspective view of the eighth embodiment of a sanitary napkin according to the present invention;

FIG. 15 is an explanatory illustration showing a testing method of an absorption test of the absorbent article;

FIG. 16 is a section of the first comparative example of the absorbent article;

FIG. 17 is a section of the second comparative example of the absorbent article;

FIG. 18 is a section of the third comparative example of the absorbent article;

FIG. 19 is a section of the conventional absorbent article; and

FIG. 20 is a section showing a condition where the sanitary napkin shown in FIG. 19 is deformed upon fitting on a user's body.

### DESCRIPTION OF THE PREFERRED EMBODIMENT

The present invention will be discussed hereinafter in detail in terms of the preferred embodiment of an absorbent article according to the present invention with reference to the accompanying drawings. In the following description, numerous specific details are set forth in order to provide a thorough understanding of the present invention. It will be obvious, however, to those skilled in the art that the present invention may be practiced without these specific details. In other instance, well-known structures are not shown in detail in order to avoid unnecessary obscurity of the present invention.

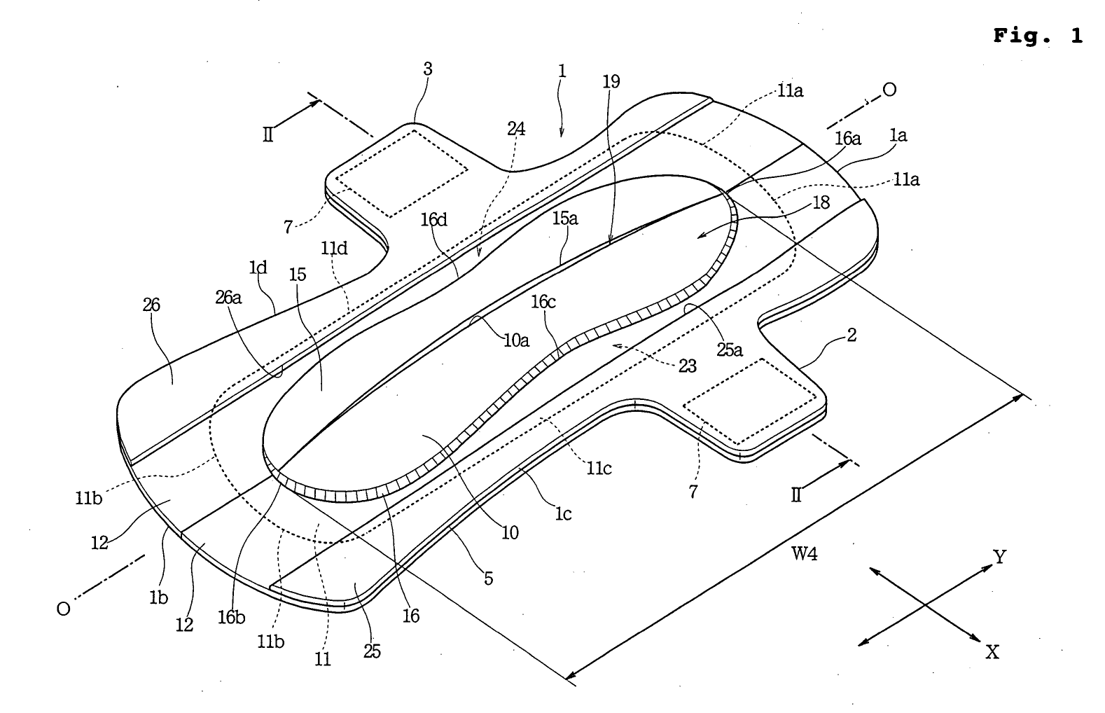

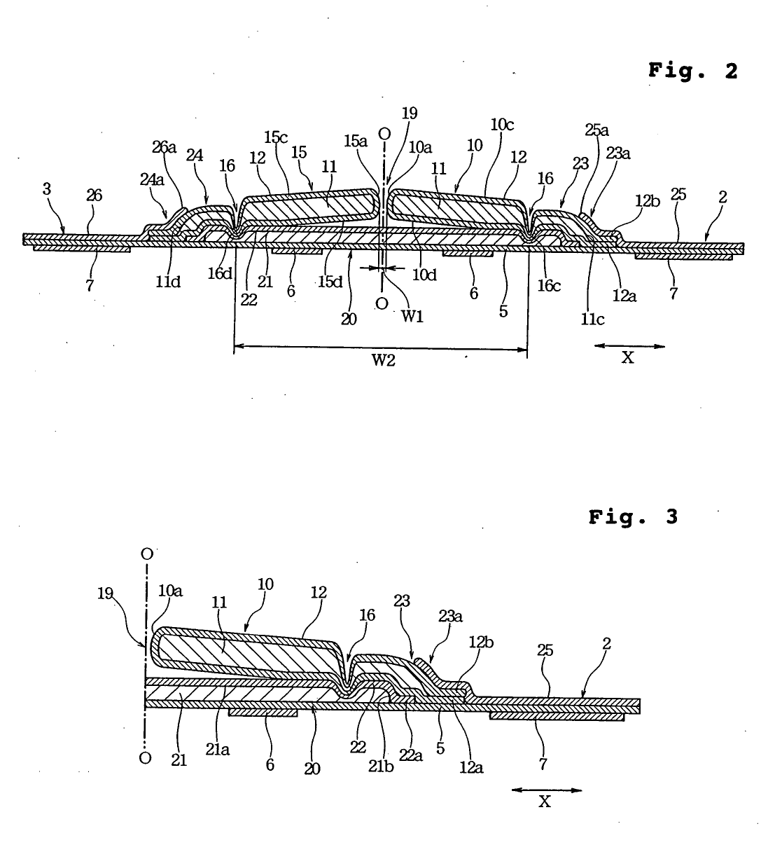

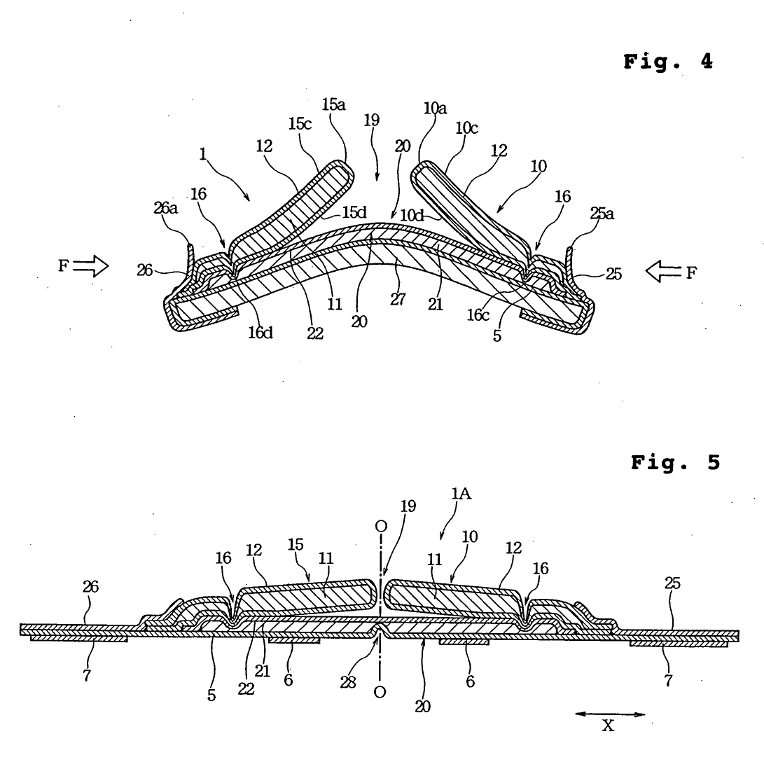

FIG. 1 is a perspective view showing a sanitary napkin 1 as the first embodiment of an absorbent article according to the present invention, shown in a condition orienting an inner surface side thereof upwardly, FIG. 2 is a cross-section taken along line II-II of FIG. 1, FIG. 3 is an enlarged section of right half of FIG. 2, and FIG. 4 is a section showing a condition where the sanitary napkin shown in FIG. 1 is deformed upon fitting on a user's body.

The sanitary napkin 1 shown in FIG. 1 is fitted on an inner surface of a crotch portion of the underwear of lady in menstrual period and mainly used for absorbing the menstrual blood.

The sanitary napkin 1 shown in FIG. 1 is longitudinally elongated shape in plan view, and has a substantially arc-shaped front edge portion 1a and a substantially arc-shaped rear edge portion 1b. A right side edge portion 1c and a left side edge portion 1d extending in longitudinal direction are curved shapes. A distance between the right side edge portion 1c and the left side edge portion 1d, namely a width dimension of the sanitary napkin 1 is greater in the rear edge portion 1b to be mainly fitted on a hip part of a wearer than that in the front edge portion 1a to be fitted mainly on a crotch part of the wearer.

At a frontwardly inclined position relative to a longitudinal center of the sanitary napkin 1, wing portions 2 and 3 are formed to extend in width or lateral direction (X direction) from the right side edge portion 1c and the left side edge portion 1d. The sanitary napkin 1 is symmetric shape relative to a center line O-O extending in longitudinal direction (Y direction). Accordingly, in FIG. 3 and subsequent drawings, a structure of only right side portion of the center line O-O is illustrated and discussed.

As shown in sections in FIGS. 2 and 3, a back sheet 5 is provided on an outer surface side of the sanitary napkin 1. The back sheet 5 is liquid impermeable and gas permeable, and is formed from a polyethylene (PE) film, polypropylene (PP) film or the like formed with fine air permeable pores. For example, the back sheet 5 is formed by blending inorganic filler, such as CaCO₃, BaSO₄ or the like to the foregoing resin material and performing drawing process for forming the film with dispersing the fine air permeable pores for enhancing air permeability. The back sheet 5 has a thickness about 15 to 50 μm. In the alternative, the back sheet 5 may also be formed from a material prepared by laminating thermoplastic resin on non-woven fabric.

The planar shape of the back sheet 5 is the same as planar shape of the sanitary napkin 1. On an outer peripheral edge of the back sheet 5, the front edge portion 1a, the rear edge portion 1b, the right side edge portion 1c, the left side edge portion 1d and the wing portions 2 and 3 are formed.

On an outer surface of the back sheet 5, pressure sensitive adhesive layers 6 are applied in longitudinally extending stripe form at center portion. On outer surfaces of the wind portions 2 and 3, pressure sensitive adhesive layers 7 are applied. In the sanitary napkin 1 before use, the pressure sensitive adhesive layers 6 and 7 are protected by covering with a releasing sheet.

On an inner surface of the back sheet 5 located on liquid receiving side, a divided absorbent element 10 and a divided absorbent element 15 extending in longitudinal direction are provided. The divided absorbent element 10 and the divided absorbent element 15 are independent of each other and are provided dividedly on both of left and right sides about the center line O-O as shown in FIGS. 1 to 3.

The divided absorbent elements 10 and 15 are symmetric structure about the center line O-O. As shown in FIGS. 2 and 4, one of the divided absorbent element 10 has a laterally inner side end portion 10a directed toward the center line O-O, an inner surface 10c directed toward the liquid receiving side and an outer surface 10d directed toward the back sheet 5. The other divided absorbent element 15 has a laterally inner side end portion 15a directed toward the center line O-O, an inner surface 15c directed toward the liquid receiving side and an outer surface 15d directed toward the back sheet 5.

Each of the divided absorbent elements 10 and 15 is formed with a longitudinally elongated divided absorbent layer 11 and a surface sheet 12 wrapping each divided absorbent layer 11. The surface sheet 12 covers the divided absorbent layer 11 over entire region of the inner surface 10c, the outer surface 10d and the inner side end portion 10a of the divided absorbent element 10. Similarly, the surface sheet 12 covers the divided absorbent layer 15 over entire region of the inner surface 15c, the outer surface 15d and the inner side end portion 15a of the divided absorbent element 15.

In each of the divided absorbent elements 10 and 15, the surface sheet 12 is bonded to the divided absorbent layer 11 by hot-melt type adhesive applied randomly so as not to interfere absorption of liquid.

The laterally inner side end portion 10a of the divided absorbent element 10 and the laterally inner side end portion 15a of the divided absorbent element 15 formed independently with each other are located in opposition at the center line O-O. As shown in FIG. 2, in a free condition where the sanitary napkin 1 is not deformed, the inner side end portion 10a and the inner side end portion 15a are lightly contacted with each other at a boundary 19. In the alternative, the inner side end portion 10a and the inner side end portion 15a may be placed in opposition in spaced apart relationship to define a gap therebetween in the boundary 19. Then, at the boundary 19, a groove portion for guiding the liquid from the inner surface toward the back sheet 5 is defined between the inner side end portion 10a and the inner side end portion 15a.

As shown in FIG. 2, a cushion layer 20 is provided on the back sheet 5 but below the divided absorbent elements 10 and 15. In the embodiment shown in FIG. 2, the cushion layer 20 serves as an auxiliary absorbent element and is formed with an auxiliary absorbent layer 21 having a given thickness and a liquid permeable sheet 22. As shown in FIG. 3, the liquid permeable sheet 22 covers an inner surface 21a and a side surface 21b of the auxiliary absorbent layer 21. An end portion 22a of the liquid permeable sheet 22 is fixedly bonded on the back sheet 5 by hot-melt type adhesive, for example. Also, an outer surface of the auxiliary absorbent layer 21 is fixedly bonded on the back sheet 5 by hot-melt type adhesive.

In the shown embodiment, a base is formed with the back sheet 5 and the cushion layer 20 overlaid thereon. The divided absorbent element 10 and the divided absorbent element 15 are provided on the base.

The divided absorbent elements 10 and 15 and the cushion layer 20 are mutually fixed at a compressed portion 16. As shown in FIG. 1, the compressed portion 16 is formed to surround a predetermined area of the inner surface of the sanitary napkin 1. Namely, the compressed portion 16 is formed into a continuous pattern of a front side compressed portion 16a, a rear side compressed portion 16b and lateral side compressed portions 16c and 16d. The region surrounded by the compressed portion 16 serves as a longitudinally elongated central absorbent region 18. The front side compressed portion 16a and the rear side compressed portion 16b are substantially arc-shaped configuration. The lateral side compressed portions 16c and 16d are inwardly curved pattern substantially analogous with the side edge portions 1c and 1d.

On both of left and right sides of the central absorbent region 18, the divided absorbent element 10 or the divided absorbent element 15 and the cushion layer 20 are fixed with each other at the lateral side compressed portion 16c and the lateral side compressed portion 16d positioned at equal distance toward left and right from the boundary 19. In the central absorbent region 18, the inner side end portion 10a of the divided absorbent element 10 and the inner side end portion 15a of the divided absorbent element 15 are formed as free ends. Within the central absorbent region 18 surrounded by the compressed portion 16, respective divided absorbent elements 10 and 15 are in free condition without bonding to the cushion layer 20.

As shown in FIGS. 1 and 2, the divided absorbent layers 11 and the surface sheets 12 wrapping the former forming the divided absorbent elements 10 and 15 extend toward outside region beyond the compressed portion 16.

As shown in FIG. 2, a right side end portion 11c of the divided absorbent layer 11 forming the divided absorbent element 10 is extended to the intermediate position between the lateral side compressed portion 16c and the right side edge portion 1c of the sanitary napkin 1. Also, end portions 12a and 12b of the surface sheet 12 wrapping the divided absorbent layer 11 are extended to the right side edge portion 1c. Similarly, a left side end portion 11d of the divided absorbent layer 11 forming the divided absorbent element 15 also is extended to the intermediate position between the lateral side compressed portion 16d and the left side edge portion 1d. Also, end portions of the surface sheet 12 wrapping the divided absorbent layer 11 are extended to the left side edge portion 1d.

As shown in FIG. 1, front end portions 11a of the divided absorbent layers 11 forming the divided absorbent elements 10 and 15 extend to intermediate positions between the front side compressed portions 16a and the front edge portion 1a, and rear end portions 11b of the divided absorbent layers 11 also extend to intermediate positions between the rear side compressed portions 16b and the rear edge portion 1b. Then, front end portions and rear end portions of the surface sheets 12 of respective divided absorbent elements 10 and 15 are located at corresponding positions of the front edge portion 1a and the rear edge portion 1b of the sanitary napkin 1.

Also, the cushion layer 20 forming the base extends to an outer peripheral region of the compressed portion 16 beyond the compressed portion 16. Namely, a peripheral edge portion of the auxiliary absorbent layer 21 forming the cushion layer 20 is extended to a position substantially corresponding to the end portions of the divided absorbent layers 11. In the alternative, the peripheral edge portion of the auxiliary absorbent layer 21 is extended to a position located slightly inclined toward center side or inner side of the end portions of the divided absorbent layers 11. Then, the liquid permeable sheet 22 has slightly greater area than the auxiliary absorbent layer 21. A peripheral edge portion of the liquid permeable sheet 22 is bonded on the back sheet 5 beyond the peripheral edge portion of the auxiliary absorbent layer 21.

In the shown embodiment, the divided absorbent elements 10 and 15 and the cushion layer 20 are heated and compressed from the inner surface side for emboss treatment. In the compressed portion 16, the divided absorbent layers 11 forming the divided absorbent elements 10 and 15 and the auxiliary absorbent layer 21 forming the cushion layer 20 are compressed toward the back sheet 5 to form a recessed groove as viewed from the inner surface side.

As shown in FIG. 2, a part of the divided absorbent element 10 is extended toward right beyond the compressed portion 16 in embossed shape. With a part of the divided absorbent layer 11 forming the divided absorbent element 10 and a part of the auxiliary absorbent layer 21, an outer absorbent portion 23 is formed. In similar manner, an outer absorbent portion 24 is formed. On the other hand, in the outer periphery side of the front side compressed portions 16a and the rear side compressed portions 16b, parts of the divided absorbent layers 11 and the auxiliary absorbent layer 21 are extended to form outer absorbent portions.

As shown in FIGS. 1 to 3, leakage preventing sheets 25 and 26 are provided to extend from positions inclined toward center side relative to outer end portions 23a and 24a of the outer absorbent portions 23 and 24 to positions corresponding to the right side edge portion 1c and the left side edge portion 1d of the sanitary napkin 1. Inner side end portions 25a and 26a of the leakage preventing sheets 25 and 26 are located overlapping with the divided absorbent layers 11. The leakage preventing sheets 25 and 26 extend in ranges from the positions overlapping with the divided absorbent layers 11 to outer edge portions of the wing portions 2 and 3 and are fixedly bonded by hot-melt type adhesive.

It should be noted that the leakage preventing sheets 25 and 26 may be free condition in a range of predetermined width from the inner side end portions 25a and 26a. On the inner side end portions 25a and 26a, longitudinally extending elastic members may be attached so that the inner side edge portions 25a and 26a of the leakage preventing sheets 25 and 26 are raised away from the divided absorbent elements 10 and 15 to enhance leakage preventing effect.

Formation of the compressed portion 16 is performed by embossing. More particularly, in the condition where the divided absorbent elements 10 and 15 are overlaid on the cushion layer 20, a roll having flat peripheral surface is fitted on back or outer surface side of the cushion layer 20, and a heating roll having a ridge portion extending along a pattern of the compressed portion 16 is urged onto the inner surface side of the divided absorbent elements 10 and 15 to heat and compress the sanitary napkin 1 between the rolls to form the compressed portion 16 in a form of the recessed groove.

At this time, by adjusting temperature and clamping pressure of the rolls depending upon material and thickness of the divided absorbent elements 10 and 15 and so forth forming the sanitary napkin 1, stiffness of the compressed portion 16 can be adjusted. The stiffness of the compressed portion 16 can be also adjusted by varying a width dimension of the ridge portion formed on the roll surface. While the compressed portion 16 is formed into recessed groove form over the entire length, compressing scores where the divided absorbent elements 10 and 15 and the cushion layer 20 are depressed to be thinner, are intermittently formed along the recessed groove in the bottom portion of the recessed groove. It should be noted that the compressing portion 16 may be a compressing score formed sequentially over the entire length.

By cutting the sanitary napkin 1 of the structure set forth above so that a part of the compressed portion 16 is located at substantially the center portion, test samples of 38 mm length (Y direction) and 25 mm width (X direction) were prepared for measuring stiffness by means of Gurley stiffness testing machine. Then, it has been found that preferred stiffness value is in a range of 4.9 to 29.4 mN. When the stiffness value is smaller than 4.9 mN, if compressing force is applied from femoral parts in a use condition where the sanitary napkin 1 is fitted on the underwear, the compressed portion 16 cannot function appropriately as bending line to cause difficulty in raising the divided absorbent elements 10 and 15 toward the user's body. On the other hand, when the stiffness value is greater than 29.4 mN, too large resistance is developed in curving the sanitary napkin 1 along the user's body to possibly form a gap between the absorbent elements of the sanitary napkin 1 and the user's body.

In each divided absorbent element 10 and 15, the thickness of the divided absorbent layers 11 at the portions between the inner side end portions 10a and 15a and the compressed portions 16 (portion of the central absorbent region 18), is preferably greater than thickness of the divided absorbent layers 11 in the portions forming the outer absorbent portions 23 and 24 located outsides of the compressing portion 16. By this, respective divided absorbent elements 10 and 15 are bulged frontwardly in the central absorbent region 18 so that the central absorbent region 18 is certainly and firmly fitted on the excretion portion of the user's body. Therefore, the user may have a feeling that the sanitary napkin 1 is firmly fitted on the user's body. Also, the excretion liquid can be easily absorbed by respective divided absorbent elements 10 and 15 to provide comfortable feeling for assurance of prevention of side leakage.

Next, an example of material forming the sanitary napkin 1 will be discussed.

The surface sheet 12 and the liquid permeable sheet 22 are preferred to have liquid permeability and high liquid permeating speeds.

For example, as material of the surface sheet 12 and the liquid permeable sheet 22, a resin film, such as polyethylene (PE) film or the like with forming large number of liquid permeable pores, may be used. In the alternative, a laminated material formed by laminating a resin firm, such as PE film or the like on a non-woven fabric having basis weight of 15 to 50 g/m² and fabricated by thermal bonding of a fibrous web of sheath-core type composite fibers having core portion of polypropylene (PP) and sheath portion of PE or core portion of polyethylene terephthalate (PET) and sheath portion of PE, and formed with liquid permeable pores, may also be used as material of the surface sheet 12 and the liquid permeable sheet 22.

Size of each individual liquid permeable pore to be formed in the surface sheet 12 and the liquid permeable sheet 22 is preferably in a range of 1.0 to 4.0 mm and area ratio of pores is preferably in a range of 25 to 50%, for example. It is also possible to apply or blend hydrophilicity providing agent, such as surface active agent, to the surface sheet 12 and the liquid permeable sheet 22 to lower surface tension and whereby to enhance wetting ability by excretion liquid for instant capturing of the excretion liquid.

On the other hand, among the surface sheets 12, at least portions located at the inner surfaces 10c and 15c of the divided absorbent elements 10 and 15 inherently contact user's skin directly. It is therefore preferred that such portions are formed of a material or in structure less irritative to user's skin in comparison with the portions located on the outer surfaces 10d and 15d of the surface sheets 12 and/or the liquid permeable sheet 22. Less irritative structure may be realized by making pitch of the liquid permeable pores smaller in the portions appearing on the inner surfaces 10c and 15c of the surface sheets 12 for reducing area of the resin film or the like to contact with the user's body or by using the resin film blended therein slip additive, Si or other material for forming fine unevenness on the surface for reducing contact area with the user's skin.

In the alternative, the surface sheet 12 and the liquid permeable sheet 22 may be formed from non-woven fabric containing hydrophilic fibers. For example, spunbonded non-woven fabric blended hydrophilic fibers, such as rayon, pulp or cotton, to synthetic fibers of PE, PP, PET or the like, having fineness in a range of 1.1 to 4.4 dtex, and adjusted basis weight in a range of 15 to 80 g/m², may be used as the surface sheet 12 and the liquid permeable sheet 22. Furthermore, the liquid permeable pores may also be formed in the non-woven fabric.

Next, the divided absorbent layer 11 is preferably formed of a material which has superior absorbing performance and can maintain elasticity even in wetted condition. For example, with foamed material containing open cell, such as cellulose sponge produced by foaming regenerated cellulose, urethane foam produced by foaming process of urethane resin and so forth, the divided absorbent layer 11 may be formed. Furthermore, the divided absorbent layer 11 may be formed by covering sliced pieces of the foamed material having thickness of 0.3 to 3.0 mm or crushed chips of the foamed material with a liquid permeable material, such as tissue paper or the like. On the other hand, in case of the foamed material having low wetting ability of liquid, such as urethane foam, wetting ability of liquid can be enhanced by impregnating hydrophilic oil.

In the alternative, the divided absorbent layer 11 may be formed with an air-laid non-woven fabric (air-laid pulp). The air-laid non-woven fabric are fabricated by stacking pulp fibers having basis weight of 50 to 200 g/m², dispersing 5 to 20% by mass of heat fusion binder, such as acryl resin or the like, relative to mass weight of the entire non-woven fabric, and binding the pulp fibers by heat.

It is also possible to further contain 5 to 30% by mass of synthetic fibers of polyethylene terephthalate (PET) or the like, having fineness of 1.6 to 4.4 dtex and fiber length of 1 to 10 mm, in air-laid non-woven fabric to attain high wet strength to easily maintain bulkiness even in a condition absorbed liquid. Furthermore, absorbing capacity of liquid can be enhanced by containing superabsorbent polymer (SAP) in a range of 5 to 50 g/m² in air-laid non-woven fabric.

In the further alternative, the divided absorbent layer 11 may be formed from a tissue paper having basis weight in a range of 15 to 30 g/m² and containing SAP in a range of 10 to 70 g/m². Also, the divided absorbent layer 11 may be formed by containing SAP in a range of 10 to 70 g/m² in a web of pulp fibers having basis weight in a range of about 50 to 300 g/m², wrapping SAP containing pulp fiber web by tissue and applying pressure for adjusting density in a range of 0.05 to 0.15 g/cm³. It is also possible to further contain 5 to 30% by mass of synthetic fibers of polyethylene terephthalate (PET) or the like having fineness of 1.6 to 4.4 dtex and fiber length of 1 to 10 mm in the SAP containing pulp fiber web, to enhance wet strength.

In the auxiliary absorbent layer 21 forming the cushion layer 20, the same material as the divided absorbent layer 11 may be used. Also, as auxiliary absorbent layer 21, relatively thin absorbent layer conventionally used as absorbent article, such as stacked body of tissue papers or SAP sheet sandwiching SAP with tissue paper, may be used.

On the other hand, the cushion layer 20 may be fibrous body having no absorbing function or the fibrous body wrapped with a sheet. The fibrous body may be through-air non-woven fabric formed from hydrophobic synthetic fibers or foamed material having low wetting ability of liquid.

The leakage preventing sheets 25 and 26 are substantially liquid impermeable and may be formed from spunbonded non-woven fabric, melt-blown non-woven fabric or stacked body of spunbonded non-woven fabric and melt-blown non-woven fabric and so forth.

FIG. 4 is a partial section of a condition where the sanitary napkin 1 is fitted on the inner surface of a crotch portion 27 of the sanitary panty.

Upon fitting, the sanitary napkin 1 is fixed on the inner surface of the crotch portion 27 by the pressure sensitive adhesive layers 6 provided on the outer surface of the back sheet 5. Furthermore, the wing portions 2 and 3 are wrapped on the outer surface of the crotch portion 27 by bending at both side portions of the crotch portion 27, and are fixedly bonded on the outer surface of the crotch portion 27 by the pressure sensitive adhesive layers 7 provided on the outer surface of the wing portions 2 and 3.

When the sanitary panty is worn and the sanitary napkin 1 is fitted on the crotch part of the wearer, compressing forces F act on the crotch portion 27 and the sanitary napkin 1 by femoral parts of wearer's body from both sides. By compressing forces F, the sanitary napkin 1 is deformed to reduce the distance between both side edge portions 1c and 1d (see FIG. 1). At this time, the sanitary napkin 1 is easily bent at the compressed portion 16 formed into recessed groove to cause bending at the lateral side compressed portion 16c and the lateral side compressed portion 16d as applied the compressing forces F to reduce a distance between the lateral side compressed portion 16c and the lateral side compressed portion 16d in lateral direction.

As a result, the central absorbent region 18 as a region defined between the lateral side compressed portion 16c and the lateral side compressed portion 16d is deformed to project toward the excretion portion of the wearer's body. Since the inner side end portions 10a and 15a of respective divided absorbent elements 10 and 15 opposing in opposition across the boundary 19 are free ends not fixed on the back sheet 5, the divided absorbent elements 10 and 15 are raised toward the wearer's body about the lateral side compressed portion 16c and the lateral side compressed portion 16d to be easily fitted onto vagina as the excretion portion of the wearer's body.

The compressed portion 16 in a form of recessed groove is formed in overlapping portion of the divided absorbent elements 10 and 15 and the cushion layer 20. The cushion layer 20 is recessed in the lateral side compressed portions 16c and 16d and recovers bulkiness at the positions adjacent the lateral side compressed portions 16c and 16d. Accordingly, the portions having recovered bulkiness and located immediately inside of the lateral side compressed portions 16c and 16d develop upward reactive force for causing raising of the divided absorbent elements 10 and 15.

On the other hand, when the compressing force F is applied and the sanitary napkin 1 is deformed to reduce distance between the lateral side compressed portion 16c and the lateral side compressed portion 16d with each other, the cushion layer 20 located between the lateral side compressed portion 16c and the lateral side compressed portion 16d is compressed from both sides to recover bulkiness to further increase reactive force to be applied to the divided absorbent elements 10 and 15 from the cushion layer 20. As set forth above, in the condition where the sanitary napkin 1 is worn on the wearer's body, respective divided absorbent elements 10 and 15 can be easily deformed to move the inner side end portions 10a and 15a in a direction away from the cushion layer 20 about the lateral side compressed portions 16c and 16d.

In the embodiment shown in FIGS. 1 to 4, the surface sheets 12 individually cover the divided absorbent layers 11 in respective divided absorbent elements 10 and 15. A single surface sheet does not cover the surfaces of two absorbent layers as in the conventional absorbent article shown in FIG. 19. Accordingly, as shown in FIG. 4, individual divided absorbent elements 10 and 15 are not mutually constrained to permit independent deformation to raise.

As set forth above, when the sanitary napkin 1 is fitted on the wearer's body and compressing force F is applied, individual divided absorbent elements 10 and 15 are independent with each other and are easily deformed so that the inner side end portions 10a and 15a are moved away from the cushion layer 20. Therefore, it is hardly cause phenomenon to cause deformation to firmly fit left and right absorbent body portions with each other as in the prior art shown in FIG. 20, thereby certainly form a gap for introducing excretion liquid between the inner side end portion 10a of the divided absorbent element 10 and the inner side end portion 15a of the divided absorbent element 15 as shown in FIG. 4.

A part of menstrual blood excreted from genital organ is applied to the surface sheet 12 in the inner surfaces 10c and 15c of the divided absorbent elements 10 and 15 firmly fitted on vagina and absorbed by the divided absorbent layers 11 through the surface sheet 12 by capillary effect of the divided absorbent layer 11.

On the other hand, as set forth above, the mutually independent divided absorbent element 10 and divided absorbent element 15 easily define the gap between the inner side end portion 10a and the inner side end portion 15a at the boundary 19. Accordingly, a part of menstrual blood may penetrate into the gap between the inner side end portion 10a and the inner side end portion 15a through the boundary 19 and be guided into the outer surfaces 10d and 15d of the divided absorbent elements 10 and 15 along the surface sheet 12 and absorbed by the divided absorbent layers 11 through the surface sheet 12 by capillary effect of the divided absorbent layer 11.

As set forth above, in the individual divided absorbent elements 10 and 15, entire surfaces of the inner surfaces 10c and 15c, the inner side end portions 10a and 15a, and the outer surfaces 10d and 15d of the divided absorbent layers 11 are independently covered with the surface sheets 12, so that surface area of the portions demonstrating absorbing function of the divided absorbent elements 10 and 15 can be substantially increased. Accordingly, liquid absorbing speed can be high and lateral propagation of the liquid can be prevented.

Furthermore, the menstrual blood applied to the boundary 19 and not absorbed by the divided absorbent elements 10 and 15 is applied to the cushion layer 20 located therebelow to be dispersed and absorbed in the cushion layer 20. Therefore, exuding of menstrual blood to the outer surface side through the back sheet 5 can be prevented.

On the other hand, since the cushion layer 20 is positioned away from the wearer's body and is not directly contacted with the wearer's skin, the menstrual blood absorbed by the cushion layer 20 will never return to the wearer's body.

Also, in the shown embodiment of the sanitary napkin 1, when menstrual blood is dispersed over all directions in the central absorbent region 18, menstrual blood is blocked at the compressed portion 16 having high density to prevent flow of menstrual blood beyond the compressed portion 16. On the other hand, even when menstrual blood is dispersed outwardly beyond the compressed portion 16, menstrual blood may be further absorbed in the outer absorbent portions 23 and 24 located outside of the compressed portion 16 to successfully prevent side leakage.

Furthermore, in the embodiment shown in FIGS. 1 to 4, since the leakage preventing sheets 25 and 26 extending in longitudinal direction are provided on outside of the compressed portion 16, even if menstrual blood is applied to the outer absorbent portions 23 and 24 beyond the compressed portion 16 and leaks outside of the outer absorbent portions 23 and 24, menstrual blood can be blocked by the inner side end portions 25a and 26a of the leakage preventing sheets 25 and 26.

On the other hand, since the divided absorbent elements 10 and 15 are surrounded by the compressed portion 16 in the predetermined area as shown in FIG. 1, menstrual blood can be concentrated within the central absorbent region 18 to successfully prevent dispersion in all directions of the sanitary napkin 1.

Hereinafter, preferred range of dimensions of the shown embodiment of the sanitary napkin 1 will be discussed. The values of the following dimensions are those in a free condition of the sanitary napkin 1 where no external force is applied.

An overall length of the sanitary napkin 1 is in a range of 180 to 350 mm, and the width dimension is in a range of 60 to 110 mm, taking the smallest dimension between the right side edge portion 1c and the left side edge portion 1d as width dimension.

In FIG. 2, a distance W1 between the inner side end portions 10a and 15a of the divided absorbent elements 10 and 15 is preferably in a range of 0 to 20 mm. When the distance W1 is smaller than 0 mm, namely when the inner side end portions 10a and 15a of the divided absorbent elements 10 and 15 overlap, one of the divided absorbent elements located at lower side becomes difficult to raise in the condition shown in FIG. 4. When the distance W1 is greater than 20 mm, it becomes difficult to contact the divided absorbent elements 10 and 15 with labia pudendi to cause difficulty in appropriately absorbing menstrual blood by the divided absorbent elements 10 and 15.

Next, as shown in FIG. 2, a minimum distance W2 between the lateral side compressed portion 16c and the lateral side compressed portion 16d is preferably in a range of 20 to 60 mm. When the distance W2 is smaller than 20 mm, width dimensions from the inner side end portions 10a and 15a of respective divided absorbent elements 10 and 15 to the lateral side compressed portions 16c and 16d become smaller than 10 mm to make area of the inner surfaces 10c and 15c of the divided absorbent elements 10 and 15 too narrow to make it difficult to absorb menstrual blood excreted from vagina only in the central absorbent region 18 and to easily cause dispersion to peripheral portion. On the other hand, in the use condition shown in FIG. 4, the divided absorbent elements 10 and 15 may not be raised to sufficient height to lower firm fitting ability of the divided absorbent elements 10 and 15 to the vagina.

On the other hand, when the distance W2 is greater than 60 mm, the distance W2 can be greater than a width of crotch part of the wearer's body to easily cause distortion of the sanitary napkin 1 when the sanitary napkin 1 is fitted on the wearer's body to make the compressed portion 16 difficult to function as bending line, and thus to cause difficulty in appropriately raising the divided absorbent elements 10 and 15 in the use condition shown in FIG. 4.

Also, a maximum distance W4 between the front side compressed portion 16a and the rear side compressed portion 16b is preferred to be in a range of ½ to ⅛ of the overall length of the sanitary napkin 1 in longitudinal direction.

FIG. 5 shows the second embodiment of a sanitary napkin 1A according to the present invention. The second embodiment of the sanitary napkin 1A is a modification of the sanitary napkin 1 of FIG. 1. It should be noted that FIG. 5 is a section taken at the same plane as that of FIG. 2. It should be noted that, in the embodiment shown in FIG. 5, like components identified by like reference numerals as in FIG. 2, are common components as the first embodiment, and disclosure for such common components will be eliminated for avoiding redundant disclosure and whereby for simplification of disclosure to facilitate clear understanding of the present invention.

In case of the sanitary napkin 1A shown in FIG. 5, the back sheet 5 is bent to project from the outer surface side to the inner surface side to compress the auxiliary absorbent layer 21 of the cushion layer 20 to form a thinned portion 28 in the cushion layer 20. The thinned portion 28 is formed to extend in longitudinal direction of the sanitary napkin 1A along the center line O-O.

It is preferred that the thinned portion 28 is formed at least in the entire area where the cushion layer 20 is provided, along the center line O-O. Formation of the thinned portion 28 can be done by providing embossing for the cushion layer 20 along the center line O-O. On the other hand, it is also possible to provide folding score along the center line O-O in place of providing the thinned portion 28 in the cushion layer 20.

When the thinned portion 28 is provided, the back sheet 5 and the cushion layer 20 positioned thereon can be easily deformed bulging toward the wearer's body upon fitting the absorbent article onto the crotch part of the wearer. As a result, the divided absorbent elements 10 and 15 can be easily fitted onto the wearer's body and easily deformed to rise toward the wearer's body.

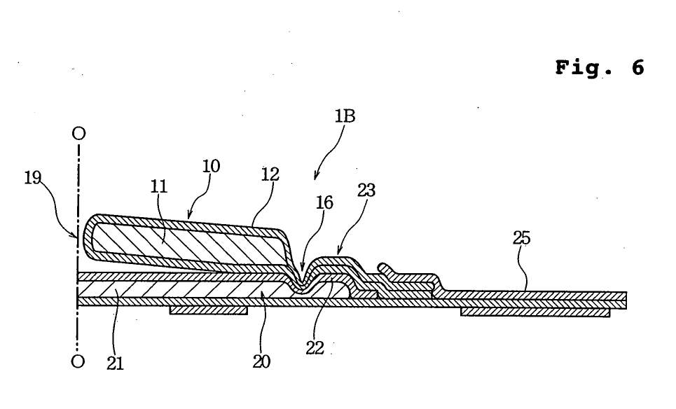

FIG. 6 is a partial section showing the third embodiment of a sanitary napkin 1B according to the present invention.

In the sanitary napkin 1B, the cushion layer 20 extends outwardly beyond the compressed portion 16, whereas the divided absorbent layer 11 forming the divided absorbent element 10 is provided only inside of the compressed portion 16. Then, the compressed portion 16 is formed by embossing the surface sheet 12 forming the divided absorbent element 10 and the cushion layer 20.

Even in the shown embodiment, when the compressing force F is applied, reaction force from the cushion layer 20 located inside of the lateral side compressed portions acts on the divided absorbent elements 10 and 15, respectively to cause deformation to raise for moving the inner side end portions 10a and 15a away from the cushion layer 20 about the compressed portion 16.

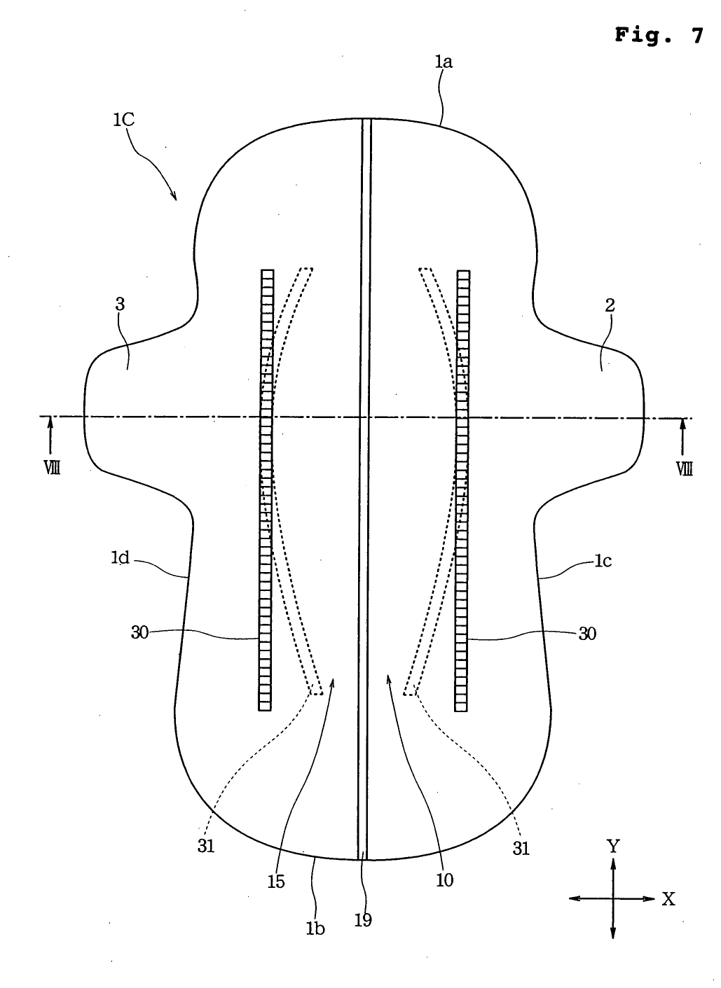

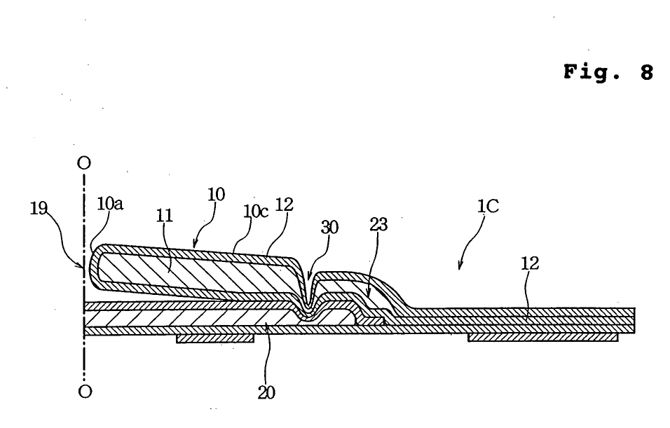

Next, FIG. 7 is a plan view showing the fourth embodiment of a sanitary napkin 1C according to the present invention. FIG. 8 is a partial section of the sanitary napkin 1C as sectioned along one-dotted line shown in FIG. 7 and viewed from a direction of VIII. It should be noted that like components identified by like reference numerals as in FIGS. 1 to 4, are common components as the first embodiment, and disclosure for such common components will be eliminated for avoiding redundant disclosure and whereby for simplification of disclosure to facilitate clear understanding of the present invention.

In the embodiment shown in FIG. 7, forming position and pattern of the compressed portion are differentiated from those of the sanitary napkin 1 shown in FIG. 1. The sanitary napkin 1C shown in FIG. 7 is provided with the divided absorbent elements 10 and 15 on both sides of the boundary 19. On both of left and right sides (in X direction in the drawing) of the boundary 19, two compressed portions 30 extending in longitudinal direction (Y direction in the drawing) are formed. In the compressed portions 30, the divided absorbent elements 10 and 15 and the cushion layer 20 are fixed with each other. In FIG. 7, the region surrounded by the compressed portion as defined in FIG. 1, is not formed. Instead, a region located between the compressed portions 30 located at both of left and right sides serves as the central absorbent region.

Since typical sanitary napkin 1C has smaller width dimension in lateral direction (X direction) than length dimension in longitudinal direction (Y direction), side leakage of menstrual blood can be caused more easily through the lateral side edge portions 1c and 1d rather than through the front edge portion 1a and the rear edge portion 1b. Accordingly, by providing the compressed portions 30 extending in longitudinal direction only on both of left and right sides, menstrual blood is prevented from reaching the lateral side edge portions 1c and 1d.

As shown in section of FIG. 8, the sanitary napkin 1C has the divided absorbent layer 11 extending outwardly from the compressed portion 30 to form the outer absorbent portion 23 at position outside of the compressed portion 30.

The surface sheets 12 of the divided absorbent elements 10 and 15 extend outwardly beyond the divided absorbent layers 11 and extend to positions corresponding to the front edge portion 1a, the rear edge portion 1b and the lateral side edge portions 1c and 1d. On the outer periphery side of the divided absorbent layers 11, two pieces of surface sheets 12 are directly stacked and bonded on the inner surface of the back sheet 5 by a hot melt type adhesive.

The compressed portions 30 may be pattern extending in straight along the longitudinal direction (Y direction in the drawing) of the sanitary napkin 1C as shown by solid line in FIG. 7, or the compressed portions may be curved pattern as shown by broken line in FIG. 7. Compressed portions 31 shown by broken line have a bow shaped pattern having maximum distance between the compressed portions 31 in the vicinity of the wing portions 2 and 3, for example. Conversely, the pattern of the compressed portions 31 may be inwardly curved pattern having minimum distance between the compressed portions 31 in the vicinity of the wing portions 2 and 3.

FIG. 9 is a plan view of the fifth embodiment of a sanitary napkin 1D according to the present invention. Also, FIG. 10 is a partial section cut at a plane shown by one-dotted line in FIG. 9 and viewed the sectioned sanitary napkin 1D from a direction of IX. It should be noted that like components identified by like reference numerals as in FIGS. 1 to 4, are common components as the first embodiment, and disclosure for such common components will be eliminated for avoiding redundant disclosure and whereby for simplification of disclosure to facilitate clear understanding of the present invention.

In the embodiment shown in FIG. 9, compressed portions 32 are formed by aggregates of a plurality of dot form compressed points 32a.

In the embodiment shown in FIG. 9, the dot form compressed points 32a are arranged in alignment in longitudinal direction (Y direction in the drawing) into two arrays which are located in spaced apart relationship with each other by a predetermined distance. In the shown embodiment, each array is formed with two rows, in each of which a plurality of the dot form compressed points 32a are aligned in longitudinal direction. It should be noted that the arrays may be formed with respectively single row of the compressed points, or with more than two rows of the compressed points. Also, in the shown embodiment, while the compressed points 32a are aligned in straight in each row, the compressed points 32a may be aligned in curved pattern. Then, the divided absorbent elements 10 and 15 and the cushion layer 20 are fixed at respective compressed points 32a, and respective compressed points 32a are recessed toward the back sheet 5.

On the other hand, in the embodiment shown in FIG. 9, the compressed points 32a are formed only on both sides of the boundary 19. It is also possible that the compressed portions 32 are formed surrounding the periphery of the divided absorbent elements 10 and 15 to define the central absorbent region.

As shown in section of FIG. 10, the divided absorbent layer 11 and the surface sheet 12 forming the divided absorbent element 10 are fixed with the cushion layer 20 at respective compressed points 32a. Therefore, similarly to the structure set forth in connection with FIG. 4, in the use condition where the sanitary napkin 1D is fitted on the underwear, the divided absorbent element 10 is easily raised to move the inner side end portion 10a away from the cushion layer 20 about the compressed points 32a.

As shown in FIG. 10, the divided absorbent layer 11 forming the divided absorbent element 10 is formed in a size extending outside of the aligned compressed points 32a to form the outer absorbent portion 23 with the divided absorbent layer 11 and the surface sheet 12 located outside of the aligned compressed points 32a. The surface sheet 12 extends beyond the outer periphery of the outer absorbent portion 23. The peripheral edge portion of the surface sheet 12 extends to the position corresponding to the outer contour of the sanitary napkin and is fixedly bonded to the back sheet 5 by hot melt type adhesive.

As shown in FIG. 9, when the compressed portions 32 are formed with the dot form compressed points 32a, regions between the compressed points 32a serve as a part of the absorbent element as shown in FIG. 10. Therefore, even when menstrual blood reaches the region where the compressed points 32a are formed, along the surface sheet 12, menstrual blood can be absorbed by the absorbent element defined between the compressed points 32a to successfully prevent side leakage of menstrual blood.

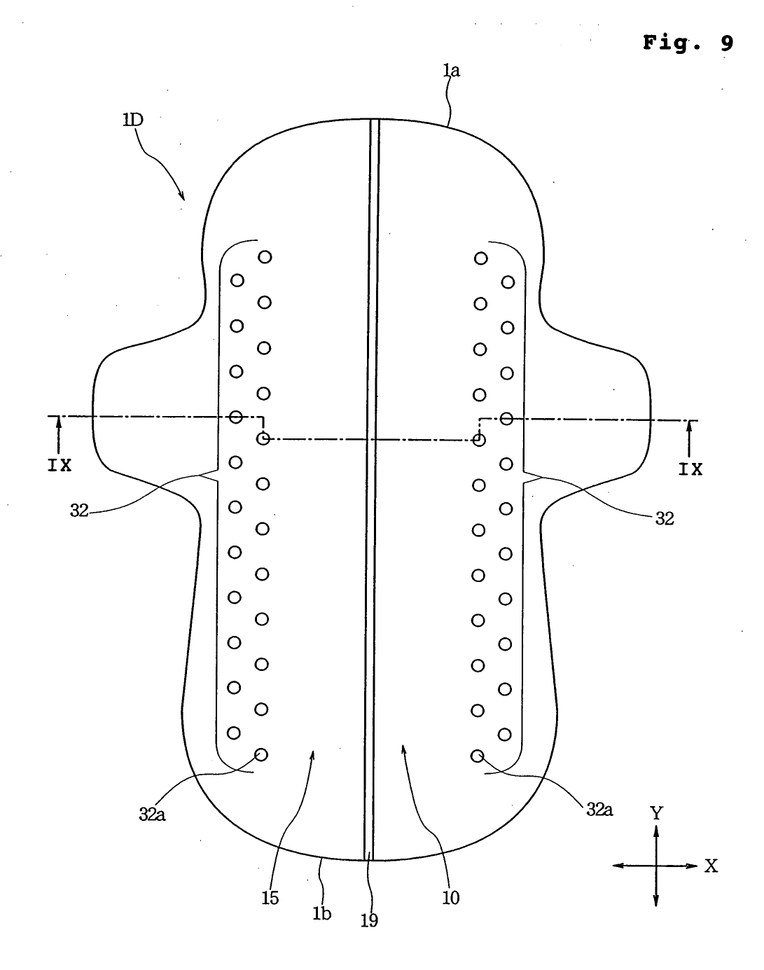

Next, FIG. 11 shows the sixth embodiment of a sanitary napkin 1E according to the present invention and is illustrated in a form of section the same as FIG. 3.

In FIG. 11, elastic members 33 extending in longitudinal direction are fixedly disposed in the inner side end portions 10a and 15a of the divided absorbent elements 10 and 15.

The elastic member 33 is formed of natural rubber, synthetic rubber such as polyurethane resin or the like, for example, and is used in a form of string or sheet. In the particular embodiment, elastic polyurethane string having fineness of about 233 to 933 dtex is stretched into about 1.05 to 1.35 times and wrapped with plastic film, non-woven fabric or the like to form the elastic member 33.

The elastic members 33 extend over the entire length of the divided absorbent elements 10 and 15 in longitudinal direction. At the front edge portion 1a and the rear edge portion 1b of the sanitary napkin 1E, the inner side end portions 10a and 15a of the divided absorbent elements 10 and 15 provided the elastic members 33 are fallen and fixedly bonded onto the surface of the cushion layers 20.

The elastic members 33 are bonded on the surface sheet 12 forming the divided absorbent elements 10 and 15 in a condition where the elastic polyurethane yarns are stretched into about 1.05 to 1.35 times. By elastic tension force of the elastic member 33, the sanitary napkin 1E is constantly biased to curve in inwardly concave with moving the front edge portion 1a and the rear edge portion 1b toward each other. As a result, the inner side end portions 10a and 15a of the divided absorbent elements 10 and 15 are certainly raised away from the surface of the cushion layer 20.

By providing the elastic member 33 as set forth above, in the use condition where the sanitary napkin 1E is fitted on the underwear, raising of the divided absorbent elements 10 and 15 is caused not only by compressing forces F applied by femoral parts of the wearer but also by elastic tension force of the elastic member 33 to certainly and firmly fit the divided absorbent elements 10 and 15 onto vaginal opening.

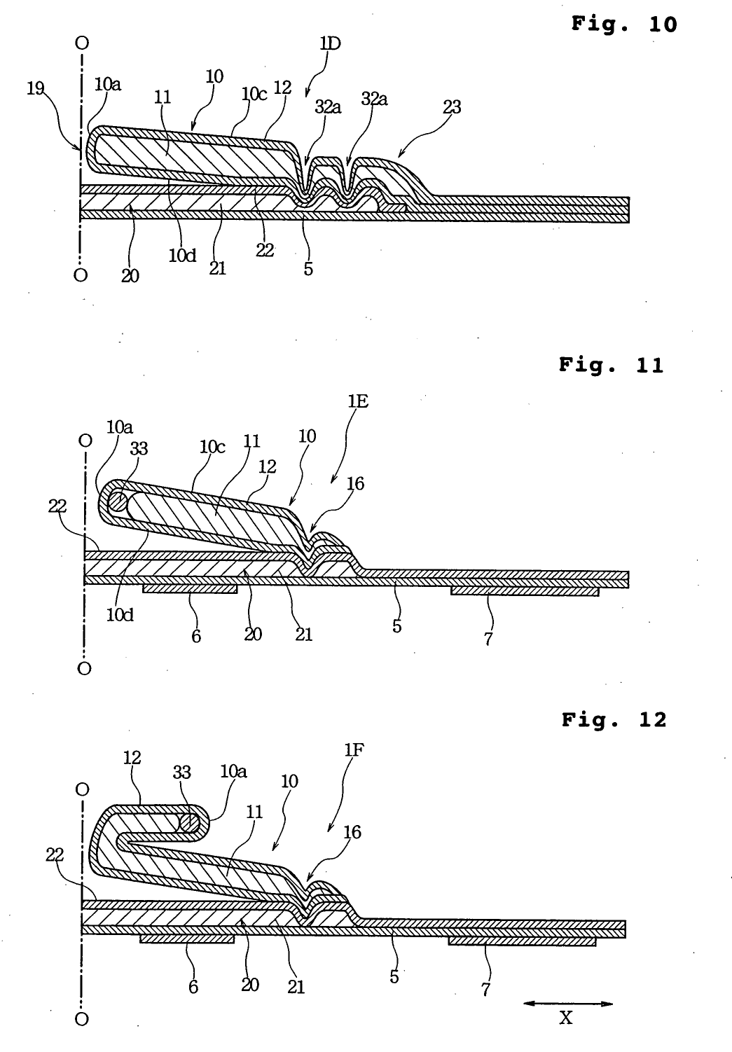

FIG. 12 shows the seventh embodiment of a sanitary napkin 1F according to the present invention and is illustrated in a form of section the same as FIG. 3.

In the embodiment shown in FIG. 12, the inner side end portions 10a and 15a of the divided absorbent elements 10 and 15 are turned back to be placed away from the boundary 19. The elastic members 33 extending in longitudinal direction are fixedly disposed in the inner side end portions 10a and 15a of the divided absorbent elements 10 and 15.

The elastic members 33 extend over entire length of the divided absorbent elements 10 and 15 in longitudinal direction. At the front edge portion 1a and the rear edge portion 1b of the sanitary napkin 1F, the inner side end portions 10a and 15a provided the elastic members 33 are turned back away from the boundary 19 and fixedly bonded on the cushion layer 20.

Similarly to FIG. 11, the elastic member 33 is bonded on the surface sheets 12 forming the divided absorbent elements 10 and 15 in a condition where the elastic members 33 are stretched into about 1.05 to 1.35 times. By elastic tension force of the elastic member 33, the sanitary napkin 1F is constantly biased to curve in inwardly concave with moving the front edge portion 1a and the rear edge portion 1b toward each other. As a result, as shown in FIG. 13, the inner side end portions 10a and 15a of the divided absorbent elements 10 and 15 are certainly raised away from the surface of the cushion layer 20 in L-shaped fashion to certainly and firmly fit the divided absorbent elements 10 and 15 onto vaginal opening.

On the other hand, at the boundary 19, a large gap is formed between the divided absorbent element 10 and the divided absorbent element 15 to transfer menstrual blood to the cushion layer 20 therethrough to absorb a large amount of menstrual blood in the cushion layer 20. Furthermore, the cushion layer 20 is placed away from the wearer's skin to hardly return absorbed menstrual blood to the wearer's skin.

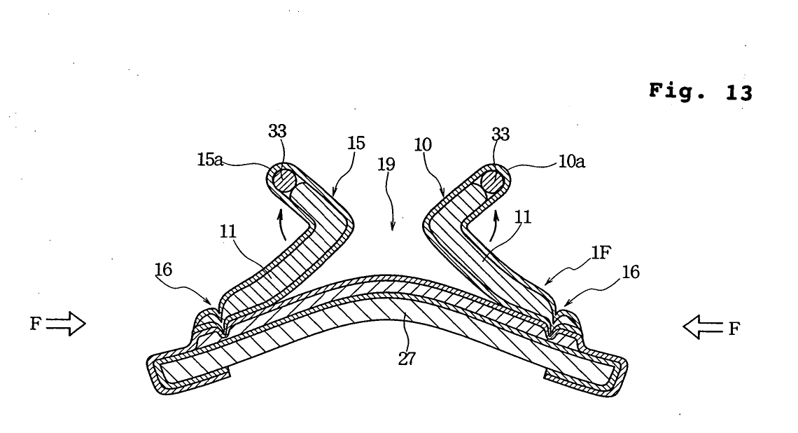

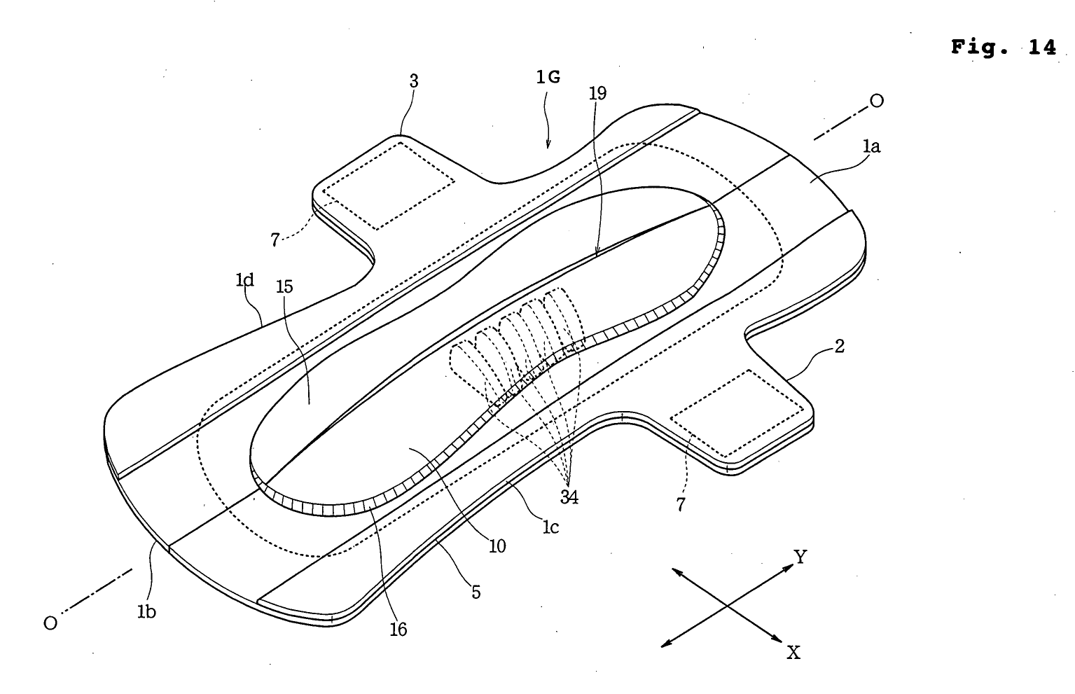

FIG. 14 is a perspective view showing the eighth embodiment of a sanitary napkin 1G according to the present invention, in similar manner to FIG. 1.

In the embodiment shown in FIG. 14, a large number of absorbent layers 34 extending in lateral direction (X direction in the drawing) (only portion of absorbent layers 34 are shown in FIG. 14) are provided in the divided absorbent elements 10 and 15. The absorbent layers 34 are aligned in longitudinal direction. Adjacent absorbent layers 34 may be contacted with each other or may be arranged with small gaps.

In the construction set forth above, in the condition where the inner side end portions 10a and 15a of the divided absorbent elements 10 and 15 are raised toward the wearer's body, menstrual blood applied to the inner side end portions 10a and 15a can be dispersed in a direction away from the inner side end portions 10a and 15a to reduce wet feeling to be provided on the wearer's skin.

It should be noted that the absorbent article according to the present invention may be applicable not only for the sanitary napkin but also for panty liner absorbing vaginal discharge, incontinence pad, absorbent assist pad for diaper, disposable diaper and so forth.

### EXAMPLE

Further preferred examples of the absorbent article according to the present invention will be discussed about performance comparing with comparative examples.

### (1) First Example

The absorbent article formed with the compressed portions 30 as illustrated by solid line in FIG. 7 is taken as the first example.

### (2) Second Example

The absorbent article having structure shown in FIGS. 12 and 13 is taken as the second example.

(Back Sheet 5)

As the back sheet 5, the liquid impermeable sheet produced by T-dye extrusion of a material containing polyethylene as primary component and having basis weight of 25 g/m², was used.

(Divided Absorbent Layer 11 and Auxiliary Absorbent Layer 21)

As the divided absorbent layer 11 and the auxiliary absorbent layer 21, superabsorbent sheet produced by forming fibrous body from hydrophilic fibers and heat-fusible fibers, and by dispersing and fixing therein superabsorbent polymer in the fibrous body, was used.

Overall basis weight of the superabsorbent sheet was set at 200 g/m², and basis weight of superabsorbent polymer contained in the superabsorbent sheet was set at 50 g/m².

(Surface Sheet 12 and Liquid Permeable Sheet 22)

As the surface sheet 12 and the liquid permeable sheet 22, through-air non-woven fabric fabricated with stable fibers of sheath-core structure consisted of polyethylene sheath and polyethylene terephthalate core and having fineness of 2 to 4 dtex and adjusted basis weight at 20 g/m², was used.

(Elastic Member)

As the elastic member, elastic polyurethane yarns having fineness of 470 dtex were fixed on the surface sheet by hot melt adhesive over a length of 70% of the absorbent layer in a condition stretched into 1.3 times.

### (3) First Comparative Example

An absorbent article shown in FIG. 16 was taken as the first comparative example.

The absorbent article was provided an absorbent layer 40 on the back sheet 5. From the portion above the absorbent layer 40 to end portions of the back sheet 5, a surface sheet 41 was provided. Different from the first and second embodiments, the absorbent article has an absorbent element not divided at both sides across the center line of the absorbent article, and a single absorbent element was provided on the back sheet 5. It should be noted that the materials of the absorbent layer 40 and the surface sheet 41 were the same as those of the examples.

### (4) Second Comparative Example

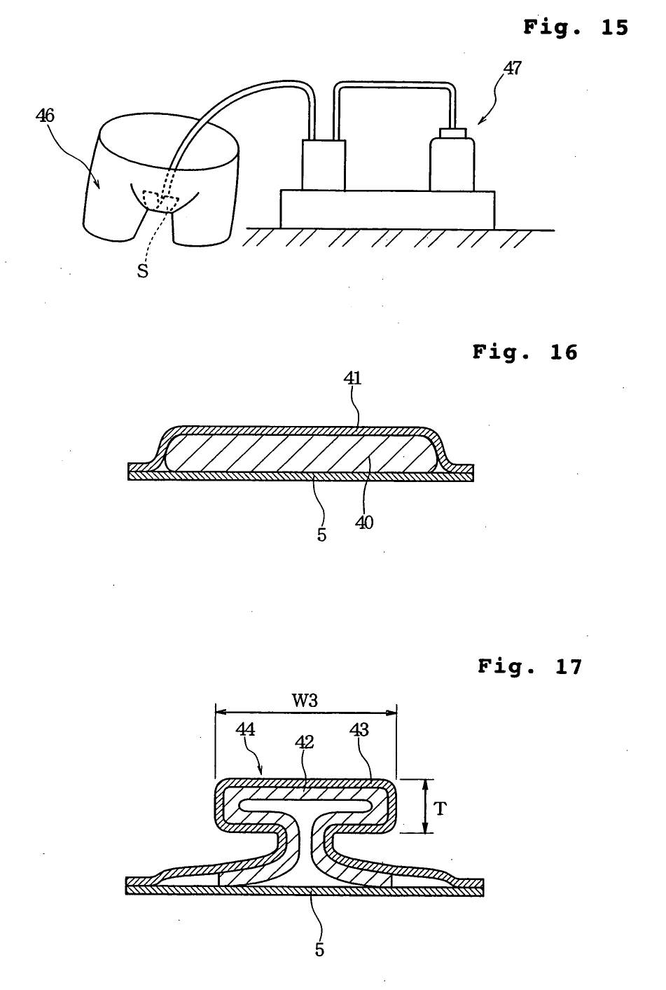

An absorbent article shown in FIG. 17 was taken as the second comparative example.

The absorbent article shown in FIG. 17 had a structure similar to that illustrated in FIG. 2 of Japanese Unexamined Patent Publication No. Heisei 9-313529.

Namely, on the back sheet 5, an absorbent layer 42 and a surface sheet 43 covering the absorbent layer 42 were provided. The absorbent layer 42 and the surface sheet 43 were folded at the center portion in lateral direction to form a ridge 44 at the center portion.

### (5) Third Comparative Example

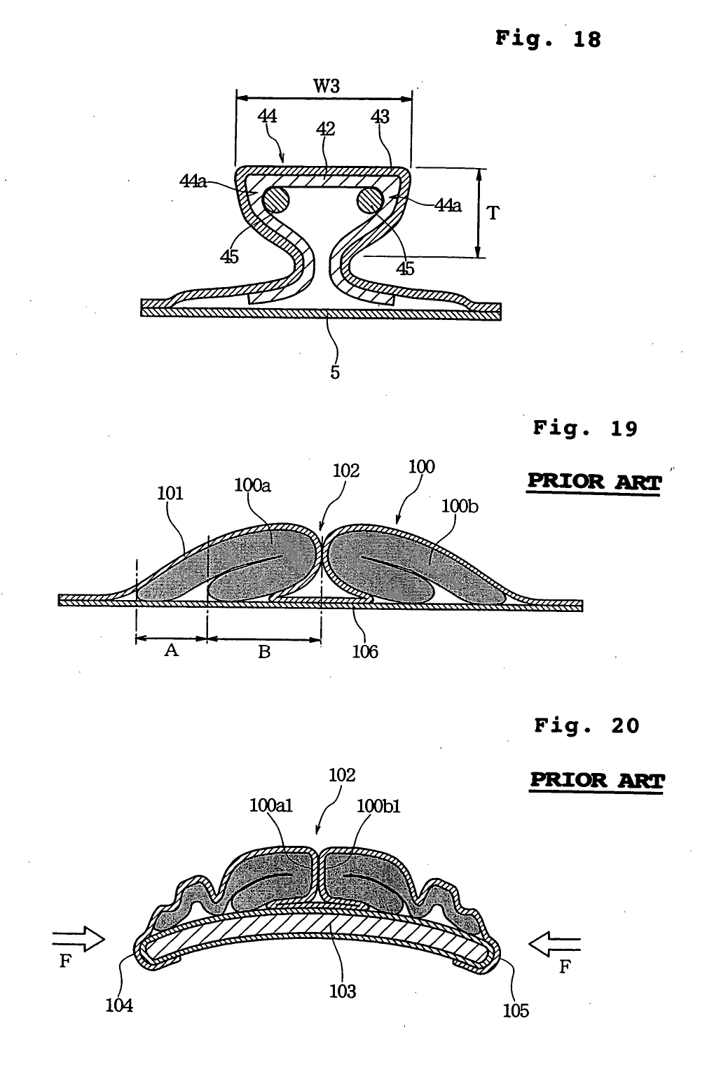

An absorbent article shown in FIG. 18 was taken as the third comparative example.

The absorbent article shown in FIG. 18 was the modification of the absorbent article shown in FIG. 17 and was not different from FIG. 17 except that elastic members 45 are disposed in the ridge 44. Both side end portions 44a of the ridge 44 disposed the elastic members 45, were fallen toward the back sheet 5 at the front edge portion and the rear edge portion of the absorbent article. By elastic tension force of the elastic member 45, the absorbent article is constantly biased to curve in inwardly concave with moving the front edge portion and the rear edge portion toward each other.

### (6) Dimension of Example and Comparative Example

In the first and second examples, distances (at the narrowest portion) between the compressed portions (30 or 16) in width direction (X direction in the drawings) were 30 mm.

On the other hand, a thickness of the sanitary napkin of the first example was set at 5 mm, a thickness excluding turned back portion of the divided absorbent elements of the second example was set at 5 mm. On the other hand, in the second example, a dimension in width direction (X direction in the drawing) of the turned back portions at the tip end portions of the divided absorbent elements 10 were set at 10 mm.

Next, width dimensions W3 of the ridges 44 of the second comparative example and the third comparative example were set at 30 mm. On the other hand, a thickness T of the respective ridges 44 was set at 5 mm.

(7) Absorbing Test Method 1

Testing method is illustrated in FIG. 15. On an anatomical model 46 simulating lady's lower body capable of discharging artificial menstrual blood, panty was set with fitting sanitary napkins S as one example of the absorbent articles of the first and second examples and the first to third comparative examples.

After setting, 3 g of artificial menstrual blood was dripped to the sanitary napkin S at a speed of 90 g/min, and subsequently left for 30 seconds, and further 4 g of artificial menstrual blood was dripped at a speed of 95 g/min.

Then, amounts of artificial menstrual blood dripped until causing leakage of artificial menstrual blood from the sanitary napkins of respective samples were measured.

(8) Result of Test

Results of test are shown in the following table 1.

TABLE 1

| | Comp. Ex | Comp. Ex | Comp. Ex | Example | Example |
|---|---|---|---|---|---|
| | 1 | 2 | 3 | 1 | 2 |
| Amount | 11 | 14 | 16 | 23 | 27 |
| Occurring | | | | | |
| Leakage (g) | | | | | |

In case of the sanitary napkins of the examples 1 and 2, amounts of artificial menstrual blood dripped until causing leakage thereof were much greater in comparison with the comparative examples of the sanitary napkins. Therefore, with the structure, in which the divided absorbent elements are provided on both sides of the center portion of the sanitary napkin and the inner side end portions of the divided absorbent elements are raised upon use as in the first and second examples, side leakage can be prevented more effectively than the structures of the first to third comparative examples.

(9) Absorbing Test Method 2

Respectively five sanitary napkins of the first example and the second comparative example were actually and alternately used by ten ladies in menstrual period. Statistics of number of ladies experienced side leakage and number of sanitary napkins caused side leakage were taken in terms of the example 1 and the comparative example 2.

(10) Result of Test

Results of test are shown in the following table 2.

TABLE 2

| | Number of People | Number of Napkins |
|---|---|---|
| | Experienced Leakage | Caused Leakage |
| Comp. Ex. 2 | 4/10 | 9/50 |
| Example 1 | 1/10 | 2/50 |

As shown in table 2, it can be appreciated that occurrence of side leakage is lower in the case where the sanitary napkin of the example 1 is used, in comparison with the case where the sanitary napkin of the comparative example 2 is used.

As set forth above, with the present invention, by dividingly forming the absorbent elements on both sides about the longitudinal center and by providing the cushion layer between the absorbent elements and the back sheet, the inner side end portions of respective absorbent elements are easily raised in use condition of the absorbent article to widen absorbing area in comparison with the conventional absorbent article and to facilitate prevention of side leakage.

On the other hand, since the absorbent article according to the present invention has high side leakage prevention effect, it is suitable for use as the sanitary napkin absorbing menstrual blood. In such case, since menstrual blood can be effectively absorbed by left and right divided absorbent element, the divided absorbent element can be formed thinner to contribute reducing thickness and size of the overall sanitary napkin. On the other hand, it becomes possible to eliminate leakage preventing walls to simplify structure of the sanitary napkin and to lower production cost.

Although the present invention has been illustrated and described with respect to exemplary embodiment thereof, it should be understood by those skilled in the art that the foregoing and various other changes, omission and additions may be made therein and thereto, without departing from the spirit and scope of the present invention. Therefore, the present invention should not be understood as limited to the specific embodiment set out above but to include all possible embodiments which can be embodied within a scope encompassed and equivalent thereof with respect to the feature set out in the appended claims.

The absorbent article according to the present invention can enhance liquid absorbing effect by dividingly forming the absorbent element, raising the opposing end portions of the divided absorbent elements in use and introducing the liquid into the gap defined between the absorbent elements and the back sheet, and can form the sanitary napkin having high liquid absorbing efficiency to realize reduction of thickness and size of the overall sanitary napkin and lowering of production cost.

1. An absorbent article provided with an absorbent element absorbing and holding a liquid, on a flat base and formed into a longitudinally elongated shape, comprising:
a cushion layer forming said base;
divided absorbent elements forming said absorbent element and dividedly formed on both sides of a center line extending in a longitudinal direction of said absorbent article;
inner side end portions of respective divided absorbent elements located in opposition across said center line; and
compressed portions located at positions away from said center line, formed by compressing said divided absorbent elements and said cushion layer together and having portions extended continuously or aligned in said longitudinal direction;
each divided absorbent element being deformable to move said inner side end portions away from said cushion layer about said compressed portions.

2. An absorbent article as set forth in claim 1, wherein each divided absorbent element has a divided absorbent layer and a liquid permeable surface sheet covering said divided absorbent layer.

3. An absorbent article as set forth in claim 1, wherein said cushion layer is an absorbent layer absorbing and holding the liquid.

4. An absorbent article as set forth in claim 1, wherein said compressed portion is formed surrounding a predetermined area of said absorbent article.

5. An absorbent article as set forth in claim 1, wherein longitudinally extending elastic members are provided in respective divided absorbent elements.

6. An absorbent article as set forth in claim 1, wherein respective divided absorbent elements are bent to place said inner side end portions toward inner surface side at positions between said inner side end portions and said compressed portion.

7. An absorbent article as set forth in claim 1, wherein a thinned portion is provided in said cushion layer along said center line.

8. An absorbent article as set forth in claim 1, wherein each divided absorbent layer is formed with a plurality of laterally extending absorbent layers.

9. An absorbent article as set forth in claim 1, which is a sanitary napkin.
